(19) United States
(12) Patent Application Publication
(10) Pub. No.: US 2012/0252073 A1
(43) Pub. Date: Oct. 4, 2012

(54) METHOD AND COMPOSITION FOR TREATING STUBBLE

(75) Inventors: Jean-Christophe Castaing, Sevres (FR); Gary Groves, Friendswood, TX (US); Ashwin Rao, Philadelphia, PA (US); Thomas E. Ruch, Voorhees, NJ (US)

(73) Assignee: RHODIA OPERATIONS, Aubervilliers (FR)

(21) Appl. No.: 13/432,106

(22) Filed: Mar. 28, 2012

Related U.S. Application Data
(60) Provisional application No. 61/516,028, filed on Mar. 28, 2011.

(51) Int. Cl. C12N 9/42; C12N 9/92; C12P 19/14; C12N 9/38; C12N 9/40; C12P 19/24; C12P 19/00; C12N 9/26

(52) U.S. Cl. 435/94; 435/209; 435/72; 435/234; 435/201; 435/207; 435/208; 435/99

(57) ABSTRACT

A method of decomposing stubble, which is crop residue left on the ground, and a composition for treating the stubble are disclosed. The method comprises treating the stubble with the composition comprising a) polysaccharide-degrading enzyme; b) wetting agent; and, optionally, c) water and allowing the treated stubble to remain on the ground and decompose.

### CROSS REFERENCE TO RELATED APPLICATIONS

This application claims the benefit of U.S. Provisional Application Ser. No. 61/516,028, filed Mar. 28, 2011, herein incorporated by reference.

### FIELD OF INVENTION

This invention relates agriculture, particularly to no-till farming, and more particularly to treating stubble crop residue methods and compositions relating to stubble and cellulose reside degradation in agricultural areas.

### BACKGROUND OF THE INVENTION

No-till farming (sometimes called zero tillage) is a way of growing crops from year to year without disturbing the soil through tillage. No-till is an emergent agricultural technique which can increase the amount of water in the soil and decrease erosion. There has been a relatively recent shift in the agricultural industry toward such “no-till” techniques. Traditionally, farmers physically plow or till their harvested land to prepare the surface to accept seeds for a new crop the following year. In the process of tilling, which is necessary to create water pathways for irrigation in the surface, the crop or cellulose reside (referred to herein as “stubble” or “crop residue is broken up and mixed into the ground allowing the residue to deteriorate more rapidly and fertilize the ground for next years crops.

Recently however, because of the increased costs of fuel required to run the machinery utilized for tilling/plowing, along with environmental concerns such as damage to the land and pollution from such machinery, there has been a push by the agricultural industry as well as from governmental agencies to develop “no-till” techniques for agricultural areas, including areas utilized for the production of crops. “No-till” techniques generally involve allowing the stubble to decompose naturally, but face significant drawbacks. One such drawback is that natural decomposition of the crop residue is slow and, as such, the agricultural surface is not optimal for growing crops the following year, which can result in a significant harvest volume decline. Another drawback to no-till agriculture which leaves stubble on the ground is that fertilizers and pesticides are retained by the stubble and larger quantities need to be used to reach the ground. Accordingly there has been significant research and interest in enhancing, increasing, and improving the decomposition of stubble.

### SUMMARY OF INVENTION

This invention comprises in one aspect a composition comprising a) a polysaccharide-degrading enzyme; b) a wetting agent; and c) optionally, water, wherein the composition is adapted to effectively degrade polysaccharide on the ground in the form of stubble.

In another aspect the invention comprises a method of decomposing stubble comprising treating the stubble with a composition comprising the polysaccharide-degrading enzyme; wetting agent; and, optionally, the water.

In some embodiments the composition consists essentially of the polysaccharide-degrading enzyme; wetting agent; and, optionally, the water, with no other active ingredients, so that the composition is cost-effective since large amounts are needed for large areas of stubble. Mixtures of two or more polysaccharide-degrading enzymes and/or two or more wetting agents may be used.

Examples of suitable polysaccharide-degrading enzymes include amylase, starch debranching enzymes, cellulases, hemicellulase, phytase, ligninases, glycosyl transferases, endoglucanases, L-arabinases, galactanases, mannanases, xylanases, pectinases, and combinations thereof. Particular polysaccharide-degrading enzymes include, by way of example, α-amylase, endo or exo-1,4- or 1,6-α-D-glucoamylase, glucose isomerase, β-amylases, α-glucosidases, and other exo-amylases, ligninase, isoamylase, pullulanase, neo-pullulanase, iso-pullulanase, amylopullulanase, cyclodextrin glycosyltransferase, exo-1,4-β-cellobiohydrolase, exo-1,3-p-D-glucanase, hemicellulase, β-glucosidase, endo-1,3-β-glucanase, endo-1,4-β-glucanase, endo-1,5-α-L-arabinase, α-arabinosidases, endo-1,4-β-D-galactanase, endo-1,3-β-D-galactanase, 1-galactosidase, α-galactosidase, endo-1,4-β-D-mannanase, 3-mannosidase, α-mannosidase, endo-1,4-β-xylanase, β-D-xylosidase, 1,3-β-D-xylanase and combinations thereof.

The wetting agent can be, for example, an anionic surfactant, a nonionic surfactant, a zwitterionic surfactant, an amphoteric surfactant or a combination thereof.

### DETAILED DESCRIPTION OF INVENTION

As mentioned above, the composition of the invention comprises a) a polysaccharide-degrading enzyme; b) a wetting agent; and c) optionally, water.

Any suitable wetting agent can be used in the composition. For example the wetting agent can be an anionic surfactant, a nonionic surfactant, a zwitterionic surfactant, an amphoteric surfactant or a combination thereof.

Some typical surfactants include but are not limited to alcohol ethoxylate, EO/PO/EO block copolymers, PO/EO/PO block copolymers, sulfo succinates including for example sodium dioctyl sulfosuccinate, phosphate esters including for example phosphate esters with ethoxylate and aliphatic chain, fluorosurfactants. Suitable anionic surfactants include alkyl and acyl taurates, alkyl and acyl sarcosinates, sulfoacetates, alkyl phosphates, alkyl phosphate esters, alkoxyl alkyl phosphate esters, acyl lactates, monoalkyl succinates and maleates, acyl isethionates, anionic derivatives of these nonionic surfactants such as the phosphate ester, ether sulfate, sulfosuccinate, and taurate types and their salts

Suitable nonionic surfactants include but are not limited to alkypolyglycosides, sorbitan fatty acid esters, aryl alkoxylates, alkoxylated fatty alcohols, alkoxylated fatty acids, alkoxylated triglycerides, alkoxy copolymers, alkoxylated fatty amines, and/or ether amines. Other suitable nonionic surfactants include but are not limited to alkoxylated mercaptans, alkoxylated carboxylic acids, and block copolymers of oxirane and methyl oxirane families.

Suitable aryl alkoxylates include phenols, which may be substituted by one or more (C4-C20)alkyl, typically (C4-C12)alkyl, or (C4-C20)aralkyl groups that are alkoxylated with up to about 100 moles (C2-C6)alkoxyl units per mole of aryl alkoxylate. Specific examples of suitable aryl alkoxylates include ethoxylated mono-, di- and tri-(phenylethyl)phenols, ethoxylated (20) nonylphenol, ethoxylated (15) octylphenol, and mixtures thereof.

Suitable alkoxylated fatty acids and alkoxylated fatty alcohols, typically (C6-C22) fatty acids and alkoxylated (C6-C22) fatty alcohols, are alkoxylated with up to about 60 moles (C2-C6)alkoxyl units per mole fatty acid or fatty alcohol. Specific examples of suitable (C6-C22) fatty alcohols or (C6-C22) fatty acids include ethoxylated (15) tridecyl alcohol, ethoxylated (7) lauryl alcohol, ethoxylated (20) oleyl alcohol, ethoxylated (15) stearyl alcohol, and mixtures thereof.

Suitable alkoxylated triglycerides include lard, tallow, peanut oil, butter oil, cottonseed oil, linseed oil, olive oil, palm oil, grapeseed oil, fish oil, soya oil, castor oil, rapeseed oil, coprah oil, coconut oil, each alkoxylated with up to about 60 moles (C2-C6)alkoxyl units per mole triglyceride. Specific examples of suitable alkoxylated triglycerides include ethoxylated (30) castor oil.

Suitable alkoxy copolymers include ethoxypropoxy copolymers, such as ethoxylated polyoxypropylene, ethoxylated/propoxylated alkylphenol block co-polymers, ethoxylated/propoxylated tristyryl phenol, and mixtures thereof.

Suitable alkylpolyglucosides, include, for example, (C8-C14)alkylpolyglucosides. Suitable alkoxylated fatty amines are alkoxylated with up to about 80 moles (C2-C6)alkoxyl units per mole of amine and include, for example, ethoxylated (15) tallow amine, ethoxylated (30) oleyl amine, and mixtures thereof.

Suitable ether amines include isopropyloxypropyl amine, isohexyloxypropyl amine, dodecyloxypropyl amine, tetradecyloxypropyl amine, linear alkyloxypropyl amine, and mixtures thereof.

Suitable cationic surfactants include, for example, babassuamidopropylkonium chloride, cocotrimonium chloride, distearyldimonium chloride, wheat germ-amidopropalkonium chloride, stearyl octyldimonium methosulfate, isostearaminopropal-konium chloride, dihydroxypropyl PEG-5 linoleaminium chloride, PEG-2 stearmonium chloride, Quaternium 18, Quaternium 80, Quaternium 82, Quaternium 84, behentrimonium chloride, dicetyl dimonium chloride, behentrimonium methosulfate, tallow trimonium chloride and behenamidopropyl ethyl dimonium ethosulfate.

Other suitable cationic surfactants include, for example, dialkyl amine derivatives. These compounds include, for example, distearyldimonium chloride, dihydrogenated palmoylethyl hydroxyethylmonium methosulfate, dipalmitoylethyl hydroxyethylmonium methosulfate, dioleoylethyl hydroxyethylmonium methosulfate, and hydroxypropyl bisstearyldimonium chloride.

Other suitable cationic surfactants include, for example, quaternary ammonium compounds of the group commonly referred to as imidazoline derivatives. Suitable imidazoline derivatives include, for example, isostearyl benzylimidonium chloride, cocoyl benzyl hydroxyethyl imidazolinium chloride, cocoyl hydroxyethylimidazolinium PG-chloride phosphate, Quaternium 32, and stearyl hydroxyethylimidonium chloride.

Amphoteric and/or zwitterionic surfactants that may be optionally included in the composition of the present invention preferably include at least one acid group, which may be a carboxylic or a sulphonic acid group. These surfactants include quaternary nitrogen and therefore are quaternary amido acids. They generally include an alkyl or alkenyl group of 3 to 18 carbon atoms and usually comply with the overall structural formula

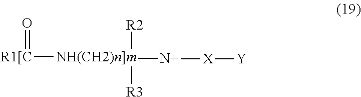

where R1 is alkyl or alkenyl of 3 to 18 carbon atoms, R2 and R3 are each independently hydrogen, alkyl, hydroxyalkyl or carboxyalkyl of 1 to 3 carbon atoms, n is 2 to 4, m is 0 to 1, X is alkylene of 1 to 3 carbon atoms optionally substituted with hydroxyl, and Y is —CO2- or —SO3-.

Suitable amphoteric and/or zwitterionic surfactants within the above general formula include simple betaines of formula

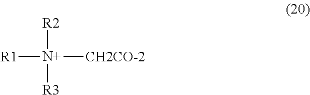

and amido betaines of formula:

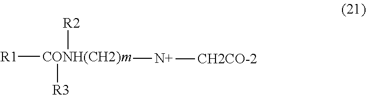

where m is 2 or 3.

In both formulae (20) and (21), R1, R2 and R3 are as defined previously in connection with formula (19). R1 may in particular be a mixture of C12 and C14 alkyl groups derived from coconut so that at least half, preferably at least three quarters, of the R1 groups have 10 to 14 carbon atoms. R2 and R3 are preferably methyl.

A further possibility is that the amphoteric and/or zwitterionic surfactant is a sulphobetaine of formula

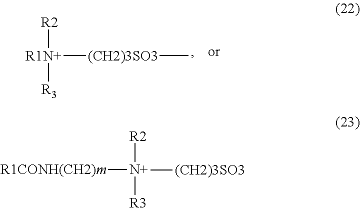

where m is 2 or 3, or variants of these in which —(CH2)3SO3 is replaced by

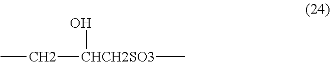

In formulas 22-24 above, R1, R2 and R3 are as defined previously in connection with formula (19).

Amphoacetates and diamphoacetates may also be used. Amphoacetates generally conform to the following formula

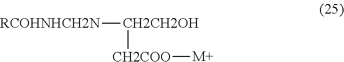

and diamphoacetates generally conform to the following formula:

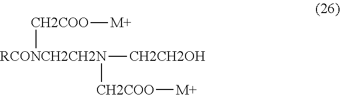

where R is an aliphatic group of 8 to 18 carbon atoms and M is a cation such as sodium, potassium, ammonium, or substituted ammonium. Sodium lauroamphoacetate, sodium cocoamphoactetate, disodium lauroamphoacetate, and disodium cocoamphodiacetate are preferred in some embodiments.

The composition described herein may further comprise water-insoluble particles or partially insoluble components, and/or one or more additional surfactants from the categories of anionic, nonionic, amphoteric, zwitterionic and cationic, or a combination of these.

The polysaccharide-degrading enzyme can be any of a variety of enzymes, including for example amylase, starch debranching enzymes, cellulases, hemicellulase, phytase, ligninases, glycosyl transferases, endoglucanases, L-arabinases, galactanases, mannanases, xylanases, pectinases, and combinations thereof.

Cellulase enzymes are a class of enzymes which can be produced by fungi, bacteria, and protozoans that catalyze the hydrolysis of cellulose. Cellulases can also be produced by other types of organisms as plants and animals. Several different kinds of cellulases are known, which differ structurally and mechanistically. In general, for cellulase activity the enzyme or enzyme complex breaks down cellulose to beta-glucose. This type of cellulase is produced mainly by symbiotic bacteria in the ruminating chambers of herbivores. Aside from ruminants, most animals (including humans) do not produce cellulase in their bodies, and are therefore unable to use most of the energy contained in plant material.

Examples of suitable enzymes other than cellulase are amylase, starch debranching enzymes, ligninases, phytase, glycosyl transferases, endoglucanases, L-arabinases, galactanases, mannanases, xylanases, and pectinases. These enzymes catalyses the breakdown of starch into sugar. Since large volumes of the composition are used in the method of the invention, only the most readily available and cost-efficient enzyme will be most suitable.

In one embodiment, no additional components are necessary in the composition therefore it is preferred that the compositions do not include any such unnecessary components. The preferred compositions consist essentially of only the enzymes, wetting agents, and optionally water and do not include any other active ingredient.

The composition in some embodiments comprises the active ingredients, the polysaccharide-degrading enzymes and wetting agents, in a ratio of about 1:100 to about 100:1 and 0 to about 100 parts water per part of the active ingredients.

In a further embodiment, certain non-surface active formulation components may also be present and these include either individually or in combination. The additional components include, but are not limited to, delaminates, buffering and/or pH control agents, fragrances, perfumes, defoamers, dyes, whiteners, brighteners, solubilizing materials, stabilizers, thickeners, corrosion inhibitors, lotions and/or mineral oils, additional enzymes (for example phytase), cloud point modifiers, preservatives, ion exchangers, chelating agents, sudsing control agents, soil removal agents, softening agents, opacifiers, graying inhibitors, stabilizers, polymers, diluents, solvents, co-solvents, preservatives, antioxidants, colorants, deposition-enhancing substances, osmolytes, organic and inorganic salts, chelating agents, fragrances, opacifiers, tackifiers, adhesives, polysaccharides and mucopolysaccharides, lignosulfonates, hydrocolloids and silicas.

Delaminates can be certain terpene-based derivatives that can include, but are not limited to, pinene and pinene derivatives, d-limonene, dipentene and oc-pinene.

The buffering and pH control agents include for example, organic acids, mineral acids, as well as alkali metal and alkaline earth salts of silicate, metasilicate, polysilicate, borate, carbonate, carbamate, phosphate, polyphosphate, pyrophosphates, triphosphates, ammonia, hydroxide, monoethanolamine, monopropanolamine, diethanolamine, dipropanolamine, triethanolamine, and/or 2-amino-2-methylpropanol.

More specifically, the buffering agent can be a detergent or a low molecular weight, organic or inorganic material used for maintaining the desired pH. The buffer can be alkaline, acidic or neutral, including but not limited to 2-amino-2-methyl-propanol; 2-amino-2-methyl-1,3-propanol; disodium glutamate; methyl diethanolamide; N,N-bis(2-hydroxyethyl)glycine; tris(hydroxymethyl)methyl glycine; ammonium carbamate; citric acid; acetic acid; ammonia; alkali metal carbonates; and/or alkali metal phosphates.

In still another embodiment, thickeners, when used, include, but are not limited to, cassia gum, tara gum, xanthan gum, locust beam gum, carrageenan gum, gum karaya, gum arabic, hyaluronic acids, succinoglycan, pectin, crystalline polysaccharides, branched polysaccharide, calcium carbonate, aluminum oxide, alginates, guar gum, hydroxypropyl guar gum, carboxymethyl guar gum, carboxymethylhydroxypropyl guar gum, and other modified guar gums, hydroxycelluloses, hydroxyalkyl cellulose, including hydroxyethyl cellulose, carboxymethylhydroxyethyl cellulose, hydroxypropyl cellulose, carboxymethylcellulose and/or other modified celluloses. In a further embodiment, the whiteners include, but are not limited to, percarbonates, peracids, perborates, chlorine-generating substances hydrogen peroxide, and/or hydrogen peroxide-based compounds. In another embodiment, the polymer is generally a water soluble or dispersable polymer having a weight average molecular weight of generally below 2,000,000.

It is understood that embodiments other than those expressly described herein come within the spirit and scope of the present claims. Accordingly, the invention described herein is not defined by the above description, but is to be accorded the full scope of the claims so as to embrace any and all equivalent compositions and methods.

What is claimed is:

1) A composition comprising:
a) a polysaccharide-degrading enzyme;
b) a wetting agent; and
c) optionally, water;
wherein the composition is adapted to degrade polysaccharide in an agricultural area.

2. The composition of claim 1 wherein the polysaccharide-degrading enzyme is selected from amylase, starch debranching enzymes, cellulases, hemicellulase, phytase, ligninases, glycosyl transferases, endoglucanases, L-arabinases, galactanases, mannanases, xylanases, pectinases, and combinations thereof.

3. The composition of claim 1 wherein the polysaccharide-degrading enzyme is selected from the group consisting of α-amylase, endo or exo-1,4- or 1,6-α-D-glucoamylase, glucose isomerase, β-amylases, α-glucosidases, and other exo-amylases, ligninase, isoamylase, pullulanase, neo-pullulanase, iso-pullulanase, amylopullulanase, cyclodextrin glycosyltransferase, exo-1,4-β-cellobiohydrolase, exo-1,3-p-D-glucanase, hemicellulase, β-glucosidase, endo-1,3-β-glucanase, endo-1,4-β-glucanase, endo-1,5-α-L-arabinase, α-arabinosidases, endo-1,4-β-D-galactanase, endo-1,3-β-D-galactanase, 1-galactosidase, α-galactosidase, endo-1,4-β-D-mannanase, 3-mannosidase, α-mannosidase, endo-1,4-β-xylanase, β-D-xylosidase, 1,3-β-D-xylanase and any combination thereof.

4. The composition of claim 1 wherein the wetting agent is an anionic surfactant, a nonionic surfactant, a zwitterionic surfactant, an amphoteric surfactant or a combination thereof.

5. The composition of claim 1 wherein the wetting agent is selected from the group consisting of alcohol ethoxylate, EO/PO/EO block copolymers, PO/EO/PO block copolymers, sulfosuccinates, phosphate esters, alkyl phosphates, alkyl phosphate esters and a mixture thereof.

6. The composition of claim 1 consisting essentially of the polysaccharide-degrading enzyme; wetting agent; and optionally, c) water.

7. A method of decomposing stubble comprising treating the stubble with a composition comprising a) polysaccharide-degrading enzyme; b) wetting agent; and, optionally, c) water.

8. The method of claim 7 wherein the polysaccharide-degrading enzyme is selected from the group consisting of amylase, starch debranching enzymes, cellulases, ligninases, glycosyl transferases, endoglucanases, L-arabinases, galactanases, mannanases, xylanases, pectinases, and combinations thereof.

9. The method of claim 7 comprising applying the composition to a no-till designated area comprising stubble on the ground and allowing the composition to react with the stubble in situ without physical removal of the stubble.

10. The method of claim 7 wherein the polysaccharide-degrading enzyme is selected from the group consisting of cellulase, hemicellulase, ligninase and a combination thereof.

11. The method of claim 7 wherein the polysaccharide-degrading enzyme is selected from the group consisting of α-amylase, endo or exo-1,4- or 1,6-α-D-glucoamylase, glucose isomerase, β-amylases, α-glucosidases, and other exo-amylases, ligninase, isoamylase, pullulanase, neo-pullulanase, iso-pullulanase, amylopullulanase, cyclodextrin glycosyltransferase, exo-1,4-β-cellobiohydrolase, exo-1,3-p-D-glucanase, hemicellulase, β-glucosidase, endo-1,3-β-glucanase, endo-1,4-β-glucanase, endo-1,5-α-L-arabinase, α-arabinosidases, endo-1,4-β-D-galactanase, endo-1,3-β-D-galactanase, 1-galactosidase, α-galactosidase, endo-1,4-β-D-mannanase, 3-mannosidase, α-mannosidase, endo-1,4-β-xylanase, β-D-xylosidase, 1,3-β-D-xylanase, and a combination thereof.

12. The method of claim 7 wherein the stubble comprises cellulose.

13. The method of claim 7 comprising spraying the composition onto the stubble.
